(19) United States
(12) Patent Application Publication
(10) Pub. No.: US 2024/0198344 A1
(43) Pub. Date: Jun. 20, 2024

(54) BLOOD CONTAINER AND MANUFACTURING METHOD THEREOF

(71) Applicant: NAN YA PLASTICS CORPORATION, TAIPEI (TW)

(72) Inventors: TE-CHAO LIAO, TAIPEI (TW); Min-Fan Chung, Taoyuan City (TW); CHING-YAO YUAN, TAIPEI (TW); CHIA-JUNG CHANG, TAIPEI (TW)

(21) Appl. No.: 18/299,720

(22) Filed: Apr. 13, 2023

(30) Foreign Application Priority Data
Dec. 15, 2022 (TW) 111148144

(51) Int. Cl. B01L 3/00

(52) U.S. Cl. CPC B01L 3/50825; B01L 2200/12; B01L 2200/16; B01L 2300/042; B01L 2300/16

(57) ABSTRACT

A blood container and a method for manufacturing the same are provided. The blood container includes a container body and an anti-fouling layer to be hydrated. The container body has an inner wall surface, and the anti-fouling layer to be hydrated covers the inner wall surface and contains a hydration polymer. A covering rate of the hydration polymer on the inner wall surface is from 50% to 100%.

### CROSS-REFERENCE TO RELATED PATENT APPLICATION

This application claims the benefit of priority to Taiwan Patent Application No. 111148144, filed on Dec. 15, 2022. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a blood container and a method for manufacturing the same, such as a blood collection tube, a centrifugal tube, a dispensing tube, and an injection tube.

### BACKGROUND OF THE DISCLOSURE

Platelet-rich plasma (PRP), also known as high-concentration platelet plasma, is obtained by centrifuging whole blood, the main active component of which is platelets. Activated PRP can release many granules, which mainly include growth factors that can stimulate cell proliferation and differentiation, and can effectively repair cellular tissues or achieve the effect of reducing inflammatory reactions. Moreover, PRP is usually prepared by collecting autologous blood, so as to avoid the risk of immunological rejection of non-autologous tissue. In recent years, PRP therapy has gradually become the main focus of regenerative medicine applications.

PRP therapy is a popular treatment, and low-cost plastic centrifugal tubes, which are usually made of a PET or PP material, are still used in the preparation process of PRP. However, traces of blood can easily cling to a tube wall due to a plastic surface of the tube wall with poor hydrophobicity and high surface roughness, resulting in a lower yield of PRP. In order to prevent drops of blood from being adhered on the centrifugal tubes, silicone oil is coated on a tube wall of each of the centrifugal tubes to improve the smoothness of the tube wall and reduce the probability of blood remained on the tube wall. However, recent studies have shown that the silicone oil can bind with proteins in blood to form silicone-containing microspheres. This may cause an inflammatory reaction in an affected area where PRP therapy is injected, and in severe cases, may cause systemic inflammation in patients.

Therefore, how to improve a blood container to effectively obtain a high yield of PRP while avoiding inflammatory reactions caused by silicone oil mixed into human body, has become one of the important issues to be solved in the related industry.

### SUMMARY OF THE DISCLOSURE

In response to the above-referenced technical inadequacies, the present disclosure provides a blood container having one or more surfaces with high hydration capacity and good antifouling properties, and a method for manufacturing the same.

In order to solve the above-mentioned problems, one of the technical aspects adopted by the present disclosure is to provide a blood container, which includes a container body and an anti-fouling layer to be hydrated. The container body has an inner wall surface, and the anti-fouling layer to be hydrated covers the inner wall surface and contains a hydration polymer. A covering rate of the hydration polymer on the inner wall surface is from 50% to 100%.

In one of the possible or preferred embodiments, a thickness of the anti-fouling layer to be hydrated ranges from 5 μm to 100 μm.

In one of the possible or preferred embodiments, the hydration polymer is a zwitterionic polymer, one molecular end of the zwitterionic polymer has an acrylate group, and another molecular end of the zwitterionic polymer has an anionic moiety or a cationic moiety.

In one of the possible or preferred embodiments, the anti-fouling layer that is hydrated has a hydrous layer formed thereon.

In one of the possible or preferred embodiments, a number average molecular weight of the zwitterionic polymer ranges from 5000 g/mol to 10000 g/mol.

In one of the possible or preferred embodiments, the anionic moiety of the zwitterionic polymer is selected from the group consisting of a phosphate group, a sulfonic acid group, and a carboxylic acid group, and the cationic moiety of the zwitterionic polymer is a quaternary ammonium group.

In one of the possible or preferred embodiments, the zwitterionic polymer includes 2-methacryloyloxyethylphosphoric choline, sulfobetaine methacrylate, carboxybetaine methyl methacrylate, or any combination thereof.

In order to solve the above-mentioned problems, another one of the technical aspects adopted by the present disclosure is to provide a method for manufacturing a blood container, which includes: providing a container body that has an inner wall surface; and treating the container body with a treatment solution including a hydration polymer, such that an anti-fouling layer to be hydrated is formed on the inner wall surface and contains the hydration polymer. A covering rate of the hydration polymer on the inner wall surface is from 50% to 100%.

In one of the possible or preferred embodiments, the treatment solution includes water, and based on a total weight of the treatment solution being 100 wt %, an amount of the hydration polymer is from 0.5 wt % to 5 wt %.

In one of the possible or preferred embodiments, the treatment solution further includes a highly volatile solvent, preferably ethanol, and based on the total weight of the treatment solution being 100 wt %, an amount of the highly volatile solvent is from 50 wt % to 99 wt %.

In one of the possible or preferred embodiments, in the step of treating the container body with the treatment solution, the container body is immersed in the treatment solution for 10 seconds to 60 seconds.

Therefore, in the blood container provided by the present disclosure, by virtue of the anti-fouling layer to be hydrated covering the inner wall surface of the container body and containing a hydration polymer, and the covering rate of the hydration polymer on the inner wall surface of the container body ranging from 50% to 100%, blood can be prevented from remaining on the inner wall surface of the container body, and biological molecules such as protein molecules can be prevented from moving closely to and being adhered on the inner wall surface of the container body.

Furthermore, the method for manufacturing the blood container provided by the present disclosure can be applied to containers commonly used for storing or processing blood, and can be performed to provide high hydration capacity and good antifouling properties to one or more surfaces of the containers within minutes.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a structural schematic view of a blood container according to embodiments of the present disclosure;

FIG. 2 is a schematic view showing a use state of the blood container according to the embodiments of the present disclosure;

FIG. 3 is another structural schematic view of the blood container according to the embodiments of the present disclosure;

FIG. 4 is a schematic enlarged view of a portion of the blood container according to the embodiments of the present disclosure; and

FIG. 5 is a flowchart of a method for manufacturing the blood container according to the embodiments of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

Unless otherwise stated, the material(s) used in any described embodiment is/are commercially available material(s) or may be prepared by methods known in the art, and the equipment or operation(s) used in any described embodiment is/are conventional equipment or operation(s) generally known in the related art.

In the present disclosure, although steps from any accompanying flowcharts may be addressed in a particular order, there is no implication or requirement with regards to the order that the steps must be executed in. In practical contexts, two or more steps of the disclosure may optionally be combined into one step, or a single step may be divided into two or more steps.

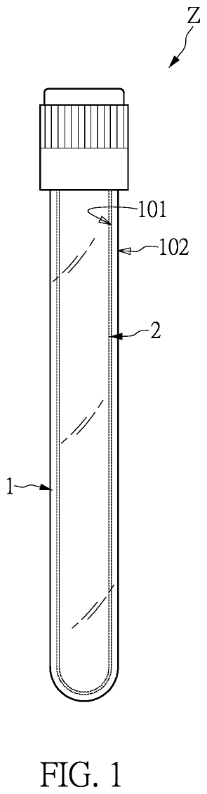

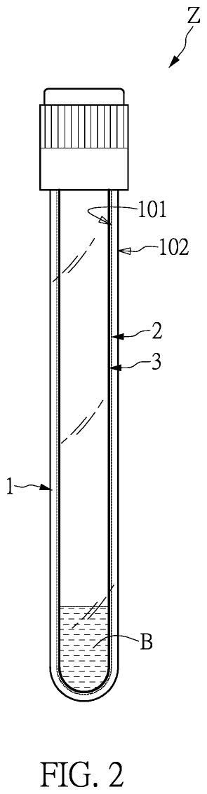

Referring to FIG. 1 to FIG. 4, embodiments of the present disclosure provide a blood container Z that includes a container body 1 and an anti-fouling layer 2 to be hydrated. The container body 1 has an inner wall surface 101, and the anti-fouling layer 2 to be hydrated covers the inner wall surface 101. Furthermore, the anti-fouling layer 2 to be hydrated contains a hydration polymer, thus having a high hydration capacity and a strong attraction to water molecules, and a covering rate of the hydration polymer on the inner wall surface 101 is from 50% to 100%. Therefore, when the blood container Z of the present disclosure is in use, a certain amount of water molecules can be bonded to the interface of the anti-fouling layer 2 to be hydrated and formed into a hydrous layer 3 (e.g., a water film), as shown in FIG. 2. Accordingly, drops of blood B can be prevented from remaining on the inner wall surface 101 of the container body 1, and biological molecules such as protein molecules can be prevented from moving closely to and being adhered on the inner wall surface 101 of the container body 1.

It should be noted that in the embodiments of the present disclosure, tubular containers such as blood collection tubes, centrifugal tubes, dispensing tubes, and injection tubes are taken as examples for describing the features of the present disclosure, but there is no restriction to the shape or structure of the container body 1. That is, the container body 1 can be other containers used for storing or processing blood. In addition, the anti-fouling layer 2 to be hydrated can have a substantially uniform thickness and chemical composition.

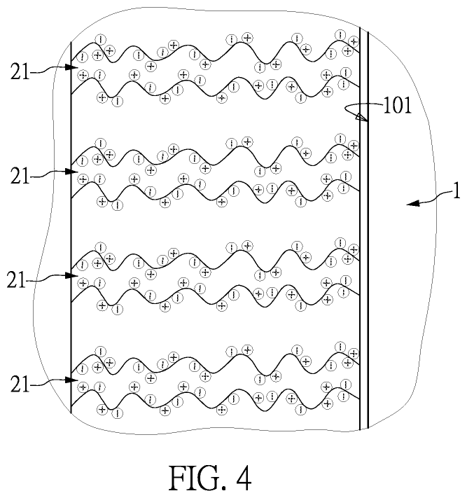

In the embodiments of the present disclosure, the hydration polymer is preferably a zwitterionic polymer, in which one molecular end of the zwitterionic polymer has an acrylate group, another molecular end of the zwitterionic polymer has an anionic moiety, and a cationic moiety is located between the acrylate group and the anionic moiety. Alternatively, one molecular end of the zwitterionic polymer has an acrylate group, another molecular end of the zwitterionic polymer has a cationic moiety, and an anionic moiety is located between the acrylate group and the cationic moiety. Preferably, a distance between a center atom of the anionic moiety and a center atom of the cationic moiety ranges from 1 carbon atom to 20 carbon atoms. Accordingly, the chemical configuration of the zwitterionic polymer on the inner wall surface 101 of the container body 1 is advantageous for the formation of the hydrous layer 3. As shown in FIG. 4, the inner wall surface 101 of the container body 1 can have a plurality of molecular chains 21 composed of the zwitterionic polymer bonded thereto. The molecular chains 21 have a sufficient length and chain flexibility so as to produce a hydration effect therebetween that promotes the structuring of water. In the zwitterionic polymer to be used for present disclosure, the anionic moiety can be selected from the group consisting of a phosphate group, a sulfonic acid group, and a carboxylic acid group, and the cationic moiety can be a quaternary ammonium group.

In order to form the hydrous layer 3 on the anti-fouling layer 2 more easily, a number average molecular weight of the zwitterionic polymer can be from 5000 g/mol to 10000 g/mol, and a thickness of the anti-fouling layer 2 to be hydrated can be from 5 μm to 100 μm.

In practice, the number average molecular weight of the zwitterionic polymer can be 5500 g/mol, 6000 g/mol, 6500 g/mol, 7000 g/mol, 7500 g/mol, 8000 g/mol, 8500 g/mol, 9000 g/mol, 9500 g/mol, or 10000 g/mol. The thickness of the anti-fouling layer 2 to be hydrated can be 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, or 100 μm.

Specifically, the zwitterionic polymer to be used for present disclosure can be 2-methacryloyloxyethyl phosphorylcholine (MPC), sulfobetaine methacrylate (SBMA), carboxybetaine methacrylate (CBMA), or any combination thereof. Specific structural formulas of 2-methacryloyloxyethyl phosphorylcholine (MPC), sulfobetaine methacrylate (SBMA), carboxybetaine methacrylate (CBMA) are shown as below; m and n are independently an integer between 1 and 20.

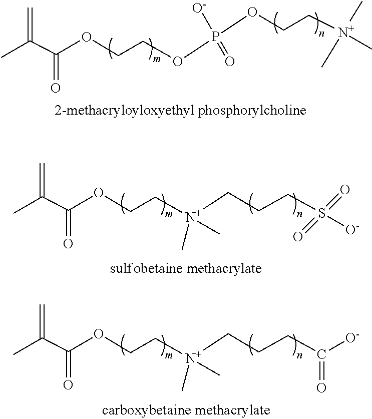

More specifically, one molecular end of each of the 2-methacryloyloxyethyl phosphorylcholine, the sulfobetaine methacrylate, and the carboxybetaine methacrylate has an acrylate group. Another molecular end of the 2-methacryloyloxyethyl phosphorylcholine is a cationic moiety (quaternary ammonium group), and another molecular end of each of the sulfobetaine methacrylate and the carboxybetaine methacrylate is an anionic moiety (sulfonic acid group or carboxylic acid group). Furthermore, in each of the 2-methacryloyloxyethyl phosphorylcholine, the sulfobetaine methacrylate, and the carboxybetaine methacrylate is 3 carbon atoms, a distance between a center atom of the anionic moiety and a center atom of the cationic moiety.

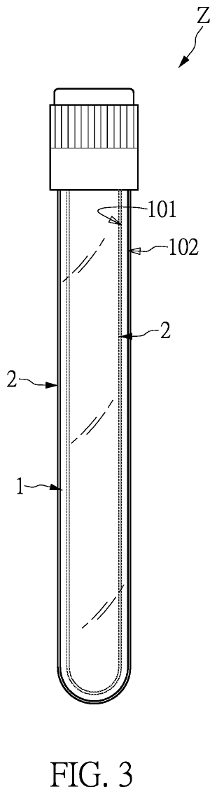

In the embodiments of the present disclosure, the anti-fouling layer 2 is formed by treating the container body 1 with a treatment solution including the hydration polymer. In a treatment process, the hydration polymer (zwitterionic polymer) can be attached to the inner wall surface 101 of the container body 1. Alternatively, the hydration polymer (zwitterionic polymer) can be attached not only to the inner wall surface 101 of the container body 1, but also to an outer wall surface 102 of the container body 1. That is, in the container body 1, only the inner wall surface 101 is covered by the anti-fouling layer 2, as shown in FIG. 1, or both the inner wall surface 101 and the outer wall surface 102 are covered by the anti-fouling layer 2, as shown in FIG. 3. The treatment solution can include the hydration polymer and water. In consideration of costs and toxicity to human cells, an amount of the hydration polymer is from 0.5 wt % to 5 wt %, based on a total weight of the treatment solution being 100 wt %.

Specifically, in the treatment process, the container body 1 is immersed in the treatment solution for 10 seconds to 60 seconds and then dried, in which the hydration polymer (zwitterionic polymer) of the treatment solution is preferably 2-methacryloyloxyethyl phosphorylcholine (molecular weight: 5000-10000), and an immersion time is preferably 30 seconds, but the present disclosure is not limited thereto. According to particular requirements, such as the evaporation rate of water, the treatment solution can further include a highly volatile solvent, preferably ethanol, and based on the total weight of the treatment solution being 100 wt %, an amount of the highly volatile solvent is from 50 wt % to 99 wt %. It should be noted that the highly volatile solvent is not particularly limited as long as it can take away water on the container body 1 while volatilizing without dissolving the zwitterionic polymer.

In the embodiments of the present disclosure, the amount of the highly volatile solvent in the treatment solution can be 50 wt %, 60 wt %, 65 wt %, 70 wt %, 75 wt %, 80 wt %, 85 wt %, 90 wt % or 99 wt %.

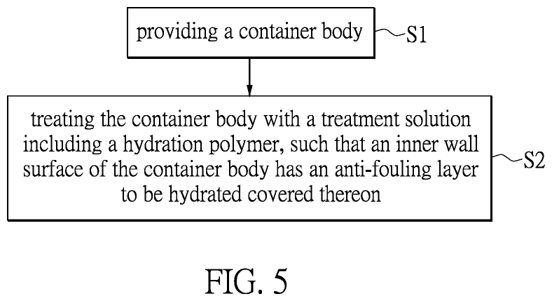

Referring to FIG. 5, the embodiments of the present disclosure further provide a method for manufacturing the blood container, which includes: providing a container body (step S1); and treating the container body with a treatment solution including a hydration polymer, such that an inner wall surface of the container body has an anti-fouling layer to be hydrated covered thereon (step S2). In the following description, specific implementation details of each step will be described with reference to FIG. 1 to FIG. 3.

In step S1, the container body 1 has a shape and structure required for storing or processing blood. For example, the container body 1 can be provided in the form of a tube and has a space that is adequate for storage purposes, but is not limited thereto.

In step S2, the anti-fouling layer 2 is formed by treating the container body 1 with the treatment solution including a hydration polymer. In a treatment process, the hydration polymer (zwitterionic polymer) can be attached to the inner wall surface 101 of the container body 1, such that the inner wall surface 101 of the container body 1 is covered by the anti-fouling layer 2, as shown in FIG. 1. Alternatively, the hydration polymer (zwitterionic polymer) can be attached not only to the inner wall surface 101 of the container body 1, but also to an outer wall surface 102 of the container body 1, such that both the inner wall surface 101 and the outer wall surface 102 are covered by the anti-fouling layer 2, as shown in FIG. 3.

Specifically, in the treatment process, the container body 1 is immersed in the treatment solution for 10 seconds to 60 seconds and then dried, in which the hydration polymer (zwitterionic polymer) of the treatment solution is preferably 2-methacryloyloxyethyl phosphorylcholine (molecular weight: 5000-10000), and an immersion time is preferably 30 seconds, but the present disclosure is not limited thereto. The treatment solution includes the hydration polymer and water. In consideration of costs and toxicity to human cells, an amount of the hydration polymer is from 0.5 wt % to 5 wt %, based on a total weight of the treatment solution being 100 wt %. The chemical composition and molecular structure of the zwitterionic polymer are described in the relevant paragraphs of the present disclosure, and will not be reiterated herein. According to particular requirements such as increasing the evaporation rate of water, the treatment solution can further include a highly volatile solvent, preferably ethanol, and based on the total weight of the treatment solution being 100 wt %, an amount of the highly volatile solvent is from 50 wt % to 99 wt %.

### [Residue Test]

Test method: immersing a PET tube (13×75 mm, 7 mL) in each of the treatment solutions of Examples 1-4 that include 2-methacryloyloxyethyl phosphorylcholine (i.e., a zwitterionic polymer) and Comparative Examples 1-3 for 30 seconds; putting the PET tubes into an oven of 50° C. to be dried for 30 minutes; filling each of the PET tubes with physiological saline or rabbit blood and then pouring the physiological saline or rabbit blood out of each of the PET tubes; and observing, by human eyes, the residual state of drops on a tube wall of each of the PET tubes. The test results are shown in Table 1 below; if the number of drops that remain is greater than five, the remaining state of drops is judged as “small amount,” and if the number of drops that are remained is less than five, the remaining state of drops is judged as “negligible amount.”

TABLE 1

| | | Remaining | |
|---|---|---|---|
| | | state of drops | Remaining |
| | | (physiological | state of drops |
| | Treatment solution | saline) | (rabbit blood) |
| Control | None | Small amount | Small amount |
| Comparative | 0.1 wt % of | Negligible | Negligible |
| Example 1 | water-based | amount | amount |
| | silicone oil | | |
| Comparative | 0.5 wt % of | No drops | No drops |
| Example 2 | water-based | | |
| | silicone oil | | |
| Comparative | 1 wt % of | No drops | No drops |
| Example 3 | water-based | | |
| | silicone oil | | |
| Example 1 | 0.1 wt % of | Negligible | Negligible |
| | zwitterionic polymer- | amount | amount |
| | containing solution | | |
| Example 2 | 0.5 wt % of | No drops | No drops |
| | zwitterionic polymer- | | |
| | containing solution | | |
| Example 3 | 1 wt % of | No drops | No drops |
| | zwitterionic polymer- | | |
| | containing solution | | |
| Example 4 | 5 wt % of | No drops | No drops |
| | zwitterionic polymer- | | |
| | containing solution | | |

### [Protein Absorption Test]

Test method: immersing a PET tube (13×75 mm, 7 mL) in each of the treatment solutions of Examples 5 and 6 that include 2-methacryloyloxyethyl phosphorylcholine (i.e., a zwitterionic polymer) and Comparative Examples 4 and 5 for 30 seconds; putting the PET tubes into an oven of 50° ° C. to be dried for 30 minutes; filling each of the PET tubes with an aqueous solution containing albumin and then shaking the PET tubes; and observing whether coagulation albumin particles are present in each of the aqueous solutions by a microscope. The test results are shown in Table 2 below.

TABLE 2

| | Treatment solution | Albumin particles |
|---|---|---|
| Comparative | 0.1 wt % of water-based | Yes |
| Example 4 | silicone oil | |
| Comparative | 1 wt % of water-based | Yes |
| Example 5 | silicone oil | |
| Example 5 | 0.5 wt % of zwitterionic | No |
| | polymer-containing solution | |
| Example 6 | 1 wt % of zwitterionic | No |
| | polymer-containing solution | |

### Beneficial Effects of the Embodiments

In conclusion, in the blood container provided by the present disclosure, by virtue of the anti-fouling layer to be hydrated covering the inner wall surface of the container body and containing a hydration polymer and the covering rate of the hydration polymer on the inner wall surface of the container body ranging from 50% to 100%, blood can be prevented from remaining on the inner wall surface of the container body, and biological molecules such as protein molecules can be prevented from moving closely to and being adhered on the inner wall surface of the container body.

Furthermore, the method for manufacturing the blood container provided by the present disclosure can be applied to containers commonly used for storing or processing blood, and can be performed to provide high hydration capacity and good antifouling properties to one or more surfaces of the containers within minutes.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A blood container, comprising:
a container body having an inner wall surface;
an anti-fouling layer to be hydrated covering the inner wall surface and containing a hydration polymer;
wherein a covering rate of the hydration polymer on the inner wall surface is from 50% to 100%.

2. The blood container according to claim 1, wherein a thickness of the anti-fouling layer to be hydrated ranges from 5 μm to 100 μm.

3. The blood container according to claim 2, wherein the hydration polymer is a zwitterionic polymer, one molecular end of the zwitterionic polymer has an acrylate group, and another molecular end of the zwitterionic polymer has an anionic moiety or a cationic moiety.

4. The blood container according to claim 3, wherein the anti-fouling layer that is hydrated has a hydrous layer formed thereon.

5. The blood container according to claim 3, wherein a number average molecular weight of the zwitterionic polymer ranges from 5000 g/mol to 10000 g/mol.

6. The blood container according to claim 3, wherein the anionic moiety of the zwitterionic polymer is selected from the group consisting of a phosphate group, a sulfonic acid group, and a carboxylic acid group, and the cationic moiety of the zwitterionic polymer is a quaternary ammonium group.

7. The blood container according to claim 3, wherein the zwitterionic polymer includes 2-methacryloyloxyethylphosphoric choline, sulfobetaine methacrylate, carboxybetaine methyl methacrylate, or any combination thereof.

8. A method for manufacturing a blood container, comprising:
providing a container body that has an inner wall surface; and
treating the container body with a treatment solution including a hydration polymer, such that an anti-fouling layer to be hydrated is formed on the inner wall surface and contains the hydration polymer;
wherein a covering rate of the hydration polymer on the inner wall surface is from 50% to 100%.

9. The method according to claim 8, wherein the treatment solution includes water, and based on a total weight of the treatment solution being 100 wt %, an amount of the hydration polymer is from 0.5 wt % to 5 wt %.

10. The method according to claim 9, wherein the treatment solution further includes a highly volatile solvent, and based on the total weight of the treatment solution being 100 wt %, an amount of the highly volatile solvent is from 50 wt % to 99 wt %.

11. The method according to claim 9, wherein in the step of treating the container body with the treatment solution, the container body is immersed in the treatment solution for 10 seconds to 60 seconds.

12. The method according to claim 9, wherein the hydration polymer is a zwitterionic polymer, one molecular end of the zwitterionic polymer has an acrylate group, and another molecular end of the zwitterionic polymer has an anionic moiety or a cationic moiety.

13. The method according to claim 12, wherein a number average molecular weight of the zwitterionic polymer ranges from 5000 g/mol to 10000 g/mol.

14. The method according to claim 12, wherein the anionic moiety of the zwitterionic polymer is selected from the group consisting of a phosphate group, a sulfonic acid group, and a carboxylic acid group, and the cationic moiety of the zwitterionic polymer is a quaternary ammonium group.

15. The method according to claim 12, wherein the zwitterionic polymer includes 2-methacryloyloxyethylphosphoric choline, sulfobetaine methacrylate, carboxybetaine methyl methacrylate, or any combination thereof.
